SEQUENCE LISTING (14 sequences)

SEQ ID NO: 1 - protein, 34 aa, Artificial Sequence
  Plumbago capensis TAT-HuR-HNS-3
YGRKKRRQRRRSPMGVDHMSGLSGVNVPGNASSG

SEQ ID NO: 2 - protein, 11 aa, Artificial Sequence
  TAT
YGRKKRRQRRR

SEQ ID NO: 3 - protein, 34 aa, Artificial Sequence
  Plumbago capensis TAT-HuR-HNS-1
YGRKKRRQRRRPNQNKNVALLSQLYHSPARRFGG

SEQ ID NO: 4 - protein, 34 aa, Artificial Sequence
  Plumbago capensis TAT-HuR-HNS-2
YGRKKRRQRRRARRFGGPVHHQAQRFRFSPMGVD

SEQ ID NO: 5 - DNA, 20 bp, Artificial Sequence
  Beta-actin primer sequence (F)
aacagtccgcctagaagcac

SEQ ID NO: 6 - DNA, 20 bp, Artificial Sequence
  Beta-actin primer sequence (R)
cgatgacatccgtaaagacc

SEQ ID NO: 7 - DNA, 17 bp, Artificial Sequence
  CXCL-2 primer sequence (F)
tcaatgcctgaagaccc

SEQ ID NO: 8 - DNA, 17 bp, Artificial Sequence
  CXCL-2 primer sequence (R)
tggttcttccgttgagg

SEQ ID NO: 9 - DNA, 21 bp, Artificial Sequence
  IL-1Beta primer sequence (F)
caggatgaggacatgagcacc

SEQ ID NO: 10 - DNA, 21 bp, Artificial Sequence
  IL-1Beta primer sequence (R)
ctctgcagactcaaactccac

SEQ ID NO: 11 - DNA, 25 bp, Artificial Sequence
  TNF-Alpha primer sequence (F)
tactgaacttcggggtgattggtcc

SEQ ID NO: 12 - DNA, 24 bp, Artificial Sequence
  TNF-Alpha primer sequence (R)
cagccttgtcccttgaagagaacc

SEQ ID NO: 13 - DNA, 20 bp, Artificial Sequence
  CCL2 primer sequence(F)
aagttgacccgtaaatctga

SEQ ID NO: 14 - DNA, 20 bp, Artificial Sequence
  CCL2 primer sequence (R)
tgaaagggaataccataaca

(19) United States
(12) Patent Application Publication
(10) Pub. No.: US 2021/0401929 A1
(43) Pub. Date: Dec. 30, 2021

(54) CELL PENETRATING SHORT PEPTIDE TAT-HUR-HNS-3 AND APPLICATION THEREOF IN INFLAMMATORY DISEASE

(71) Applicant: Northeast Normal University, Changchun (CN)

(72) Inventors: Xueqing BA, Changchun (CN); Yueshuang KE, Changchun (CN); Ke WANG, Changchun (CN)

(21) Appl. No.: 17/142,051

(22) Filed: Jan. 5, 2021

(30) Foreign Application Priority Data
Jun. 29, 2020 (CN) 202010605471 .0

(51) Int. Cl. A61K 38/16

(52) U.S. Cl. CPC A61K 38/16

(57) ABSTRACT

The present disclosure relates to a cell penetrating short peptide TAT-HuR-HNS-3 and application thereof in inflammatory disease, wherein the amino acid sequence of cell penetrating short peptide TAT-HuR-HNS-3 for inflammatory diseases caused by elevated inflammatory factors is YGRKKRRQRRR-SPMGVDHMSGLSGVNVPGNASSG, the inflammatory diseases caused by elevated inflammatory factors include lung inflammation, asthma, pollen allergy, and nephritis; TAT-HuR-HNS-3 may specifically inhibit the interaction of HuR-PARP1/HuR-HuR and the expression of inflammatory factors, and has no effect on cell proliferation and survival. This provides a safety guarantee for the drug development of cell penetrating peptides, and is a new method applied to the treatment of inflammatory diseases.

### CROSS REFERENCE TO RELATED APPLICATION

This application claims the priority of Chinese Patent Application No. 202010605471.0, entitled “Cell penetrating short peptide TAT-HuR-HNS-3 and application thereof in inflammatory disease” filed with China National Intellectual Property Administration on Jun. 29, 2020, which is incorporated herein by reference in its entirety.

### TECHNICAL FIELD

The disclosure belongs to the medical field, and in particular to a cell penetrating short peptide TAT-HuR-HNS-3 and application thereof in inflammatory disease.

### BACKGROUND ART

Gene expression regulation of eukaryotes is complex, and regulation of post-transcriptional level plays an important role in the accurate and rigorous gene expression. The mRNA degradation determines the mRNA stability, which affects protein expression^{[1]}. The amount of mRNA expression determines the content of protein to be translated, which makes even a small change in mRNA cause a huge change in protein content. The mRNA of inflammatory factor and cytokine are usually low expressed in cells, and only when the cells under stress condition will cause their accumulation, which translates into cytokines that help cells respond to outside stimulus. After the whole process subsides, these mRNAs will also be degraded. If there is an error in a certain link in this process, so that these inflammation-related mRNAs are not normally degraded, it will cause the overexpression of cytokines, which will cause cell overreaction, leading to some autoimmune diseases and chronic inflammation. Even the occurrence of cancer^{[1-3]}. Approximately 8% of mRNA in mammalian cells contains AU-rich element (ARE) in 3′untranslated region, which is an AU-rich sequence. The mRNAs containing ARE are easily degradable mRNAs^{[4]}. The mRNA stability is regulated by RNA-binding proteins. Most ARE binding proteins mediate the degradation of mRNA, however HuR is one of the few RNA-binding proteins that stabilize mRNA, which has received widespread attention.

As an RNA-binding protein that plays an important role in the post-transcriptional regulation of genes, Human antigen R (HuR) may play an important role in the production and development of diseases such as cancer and inflammation. In macrophage, endothelial cells, intestinal epithelial cells, colon cancer cells, gastric cancer cells and cervical cancer cells, HuR binds to the mRNA of the pro-inflammatory cytokines IL-8, TGF-β and IFN-γ, which enhancing the expression of these factors^{[5-7]}. In inflammation-related diseases, such as rheumatoid arthritis, HuR promotes the expression of TNF-α^{[8, 9]}. In rheumatic cartilage and osteoarthritis, HuR is involved in the regulation of COX-2^{[10]}. In inflammatory bowel disease, HuR up-regulated the expression of COX-2 in intestinal epithelial cells^{[11]}. In chronic inflammatory disease asthma, HuR is involved in the up-regulation of TNF-α, GM-CSF and other factors^{[12]}. It is currently known that HuR can significantly promote the inflammatory response by interacting with the mRNA of the pro-inflammatory cytokines.

The function of HuR is mainly achieved through protein modification. In the previous work of our laboratory, we found that HuR can undergo a poly ADP-ribosylation (PARylation) modification under stress condition. PARP1 is the most important member of the PARP family and participates in the most PARylation modifications in the cell^{[13]}. When the cells were under stress condition, PARP1 interact with HuR and modify HuR by PARylation, and then HuR's nucleocytoplasmic shutting is promoted^{[14]}. In addition, studies have confirmed that HuR protein can be oligomerized, i.e. HuR-HuR interaction, and the oligomerization is necessary for HuR to stabilize mRNA^{[15, 16]}. Our previous work shows that PARylation modification can also promote the HuR-HuR interaction. Our previous experiments have proved that the nucleocytoplasmic shuttling sequence (HNS) region of HuR can mediate the interaction between HuR-PARP1 and HuR-HuR^{[14, 17]}.

Cell penetrating peptides (CPPs) are a large class of sequences composed of 10-30 amino acids, and also known as protein transduction regions or transduction peptides. So far, the CPPs that have been discovered include Pep-1, pVEC, MAP, pAntp, Transportan, Polyarginines, TAT, etc. Among them, TAT is a very classic cell penetrating peptide, which has a high efficiency of penetration^{[18, 19]}. TAT contains 11 amino acids, the amino acid sequence of which is YGRKKRRQRRR (SEQ ID NO.2). TAT can be located in the nucleus and cytoplasm, and has been widely selected as a delivery tag for biological macromolecules^{[20]}.

### SUMMARY OF THE APPLICATION

The purpose of the present disclosure is to provide a cell penetrating short peptide TAT-HuR-HNS-3 and application thereof in inflammatory disease caused by the increase of inflammatory factors. The amino acids sequence of HuR-HNS-3 may specifically inhibit the interaction of HuR-PARP1/HuR-HuR. The cell penetrating peptides TAT is used as a carrier to deliver the amino acid sequence (short peptide) into the cell, and the cell penetrating peptide TAT-HuR-HNS-3 may inhibit HuR-PARP1/HuR-HuR interaction; the cell penetrating peptide has no effect on cell proliferation and survival, and effectively inhibit the expression of inflammatory factors CCL2, CXCL2, TNF-α, IL-1β, which suggest the cell penetrating peptide TAT-HuR-HNS-3 may be used to treat inflammatory diseases.

The present disclosure is realized as follows: a cell penetrating short peptide TAT-HuR-HNS-3, wherein the amino acid sequence of the cell penetrating short peptide TAT-HuR-HNS-3 for inflammatory diseases caused by the increase of inflammatory factors is as follows: YGRKKRRQRRR-SPMGVDHMSGLSGVNVPGNASSG (SEQ ID NO.1).

In one embodiment, the amino acid sequence containing HuR-HNS-3 is used in the drug treating inflammatory diseases caused by the increase of inflammatory factors, wherein the inflammatory diseases comprising lung inflammation, asthma, pollen allergy, and nephritis.

In one embodiment, a delivery carrier connected to the amino acid sequence containing HuR-HNS-3 includes Pep-1, pVEC, MAP, pAntp, Transportan, Polyarginines.

In one embodiment, nanometer or other small molecule materials are selected as a delivery carrier of the amino acid sequence containing HuR-HNS-3.

In some embodiments, the TAT-HuR-HNS-3 or HuR-HNS-3 short peptide connected by other carriers or drugs containing HuR-HNS-3 and their application in inflammatory diseases of the lungs and other tissues caused by elevated inflammatory factors are provided.

The advantageous effects of the present disclosure:

1. The Cell Penetrating Peptide TAT-HuR-HNS-3 May Inhibit the Interaction of HuR-PARP1/HuR-HuR.

In this experiment, TAT is selected as a delivery tag. TAT is derived from the HIV virus Tat protein, with a total of 11 amino acids. The sequence has high efficiency of penetration than the full-length of Tat protein, and it is located in the nucleus and cytoplasm, and has been widely selected as a delivery tag for biological macromolecules. The HNS region of HuR is divided into three overlapping parts, and the N-terminus is connected to the cell penetrating peptide TAT as a delivery carrier: TAT, TAT-HuR-HNS-1 (SEQ ID NO.3), TAT-HuR-HNS-2 (SEQ ID NO.4), TAT-HuR-HNS-3 (as shown in FIG. 1). The whole cell lysate of human renal epithelial cells HEK293 is extracted as the free phase, and incubated with GST and GST-HuR reduced glutathione 4B beads at 4° C. at stationary phase. TAT, TAT-HuR-HNS-1, TAT-HuR-HNS-2 and TAT-HuR-HNS-3 are added to interfere with the interaction between HuR and PARP1, and the working concentration of cell penetrating peptide is 25 μM. The result of western blot detection shows that the short peptide TAT-HuR-HNS-3 can significantly inhibit the interaction of HuR-PARP1 (as shown in FIG. 2).

Furthermore, the GST, GST-HuR reduced glutathione 4B beads and the induced and purified His-HuR protein are incubated at 4° C. at stationary phase, and TAT, TAT-HuR-HNS-1, TAT-HuR-HNS-2 and TAT-HuR-HNS-3 are added to interfere with the interaction of HuR-HuR, and the working concentration of cell penetrating peptide is 25 μM. The result of western blot shows that TAT-HuR-HNS-3 can significantly inhibit the interaction between HuR and HuR (as shown in FIG. 3).

Furthermore, in order to explore the effect of the cell penetrating peptide on the interaction of HuR-PARP1 and HuR-HuR within the cell. The results of protein Co-immunoprecipitation and western blot show that the interaction of HuR-PARP1 (as shown in FIG. 4) and HuR-HuR (as shown in FIG. 5) are significantly inhibited in the cells added with TNA-HuR-HNS-3 peptide.

2. The Cell Penetrating Peptide TAT-HuR-HNS-3 Inhibits the Expression of Inflammatory Factors at the Cell Level.

In macrophage, endothelial cells, intestinal epithelial cells, colon cancer cells, gastric cancer cells and cervical cancer cells, HuR may bind to the mRNA of the pro-inflammatory cytokines TNF-α, IL-6, IL-8, TGF-β and IFN-γ, which enhancing the expression of these factors. The results of preliminary experiments have found that C—X—C cell chemokines, interleukins, and tumor necrosis factor families belong to the target mRNA regulated by HuR. The expression of cytokines such as TNF-α, IL-10, and CXCL2 is detected in murine lung epithelial cells MLE12 and murine macrophage RAW264.7. The cells are pretreated with cell penetrating peptide TAT-HuR-HNS-3 0.5 h in advance, and then the cells are stimulated with LPS. After 1 h, RNA of different stimulated groups is extracted and reversely transcribed, and the cDNA is selected for PCR experiment to detect the expression different inflammatory factors. Olaparib as a PARP inhibitors, may inhibit the expression of inflammatory factors, and is used as a positive control. The results show that TAT-HuR-HNS-3 can inhibit the mRNA levels of inflammatory factors CXCL2 (as shown in FIG. 6) and TNF-α (as shown in FIG. 7) in murine lung epithelial cells MLE12.

In addition, the effect of cell penetrating peptide TAT-HuR-HNS-3 on the expression of inflammatory factors in murine macrophages RAW264.7 is detected. TNF-α is used to stimulate murine macrophages RAW264.7, and TAT-HuR-HNS-3 short peptide is added. After 1 hour, the RNA from different stimulated groups is extracted and reversely transcribed, then cDNA is selected for PCR experiments to detect the expression of different inflammatory factors. The results show that TAT-HuR-HNS-3 short peptide also may inhibit the mRNA levels of inflammatory factors CXCL2 (as shown in FIG. 8), IL-10 (as shown in FIG. 9), and TNF-α (as shown in FIG. 10).

At the same time, the experiment is repeated in murine macrophage RAW264.7. The murine macrophage RAW264.7 is stimulated with LPS, and TAT-HuR-HNS-3 short peptide is added. After 1 h, the RNA from different stimulation groups is extracted. The cDNA obtained by reverse transcription is selected for PCR experiments to detect the expression of different inflammatory factors. The results show that TAT-HuR-HNS-3 short peptide also inhibit the mRNA levels of inflammatory factors CXCL2 (as shown in FIG. 11), IL-1β (as shown in FIG. 12), and TNF-α (as shown in FIG. 12). The results show that TNF-α, IL-1β, CXCL2 and other inflammatory response factors are up-regulated after being stimulated with TNF-α or LPS. Pretreatment of the cell with cell penetrating peptide TAT-HuR-HNS-3 may reduce the mRNA level.

3. The Cell Penetrating Peptide TAT-HuR-HNS-3 Inhibits Murine Lung Inflammation

In order to further confirm that the cell penetrating peptide TAT-HuR-HNS-3 has the effect of inhibiting inflammation, the lung inflammation mice model is established by giving LPS via intranasal administration. The lung inflammation mice model in different groups are compared, i.e. Saline (normal saline) group, LPS group, TAT-HuR-HNS-3 group and LPS+TAT-HuR-HNS-3 group. The alveolars lavage fluid of mice treated with different stimulation are smeared and stained with Wright-Giemsa. The alveolars lavage fluid of mice with LPS stimulation contains a large number of neutrophils. Pretreating the mice with cell penetrating peptide TAT-HuR-HNS-3 significantly reduce the number of neutrophils (as shown in FIG. 14). Then the lungs of mice treated with Saline (normal saline) group, LPS group, TAT-HuR-HNS-3 group and LPS+TAT-HuR-HNS-3 group are ground, RNA is extracted, and real-time PCR is performed to detect the levels of inflammatory factors CCL2, CXCL2, IL1β and TNFα mRNA after reverse transcription. As shown in FIG. 15, treating mice with cell penetrating peptide TAT alone do not cause inflammation. Inflammation occurs in the lungs of mice after being stimulated by LPS for 1 hour, and the mRNA expression of CCL2, CXCL2, IL-1β, and TNF-α is up-regulated. Compared with the LPS group, the expression of inflammatory factors of in mice pretreated with the cell penetrating peptide TAT-HuR-HNS-3 is significantly down-regulated. The cell penetrating peptide TAT-HuR-HNS-3 can inhibit lung inflammation of mice.

### BRIEF DESCRIPTION OF THE DRAWINGS

FIG. 1 is a photograph showing the amino acid sequence of the cell penetrating peptide in the HuR-HNS region.

FIG. 2 is a photograph showing the short peptide TAT-HuR-HNS-3 can inhibit the interaction between HuR and PARP1. The whole cell lysate of human renal epithelial cells HEK293 is extracted as the free phase, and incubated with GST and GST-HuR reduced glutathione 4B beads at 4° C. in stationary phase for 3 h. TAT, TAT-HuR-HNS-1, TAT-HuR-HNS-2 and TAT-HuR-HNS-3 are added to interfere with the interaction between HuR and PARP1, and the working concentration of cell penetrating peptide is 25 μM. The result of western blot detection shows that the short peptide HuR-HNS-3 can significantly inhibit the interaction of HuR-PARP1.

FIG. 3 is a photograph showing that His-HuR, GST and GST-HuR reduced glutathione 4B beads are incubated at 4° C. f at stationary phase or 3 h, and HuR-HNS-1, HuR-HNS-2, HuR-HNS-3 are added to interfere with HuR-HuR. The working concentration of cell penetrating peptide is 25 μM. The result of western blot shows that TAT-HuR-HNS-3 can inhibit the interaction of HuR-HuR.

FIG. 4 is a photograph showing TAT, TAT-HuR-HNS3 are added to the cell culture medium 0.5 h in advance, and then the whole cell lysate is extracted after TNF-α has stimulated MLE12 cells for 5 hours, and HuR antibody is selected to Co-immunoprecipitation. The western blot is performed to detect the binding of PARP1 to HuR. The working concentration of cell penetrating peptide TAT, TAT-HuR-HNS-3, is 25 μM.

FIG. 5 is a photograph showing TAT, TAT-HuR-HNS3 are added to the cell culture medium 0.5 h in advance, and then the whole cell lysate is extracted after TNF-α has stimulated HEK293 cells for 1 hour, and GFP antibody is selected to Co-immunoprecipitation. The western blot is performed to detect the binding of GFP-HuR to HuR. The working concentration of cell penetrating peptide TAT, TAT-HuR-HNS-3 is 25 μM.

FIG. 6 is a photograph showing TAT, TAT-HuR-HNS-3 and olaparib are added to the cell culture medium 0.5 h in advance. The expression of CXCL2 mRNA is detected by Realtime PCR after LPS has stimulated MLE12 cells for 1 hour. The working concentration of cell penetrating peptide is 25 μM. **P<0.01, the effect of cell penetrating peptide on the expression of inflammatory factor mRNA is compared with that of LPS stimulation group, n=3.

FIG. 7 is a photograph showing TAT, TAT-HuR-HNS-3 and olaparib are added to the cell culture medium 0.5 h in advance. The expression of TNF-α mRNA is detected by Realtime PCR after LPS has stimulated MLE12 cells for 1 hour. The working concentration of cell penetrating peptide is 25 μM. **P<0.01, the effect of cell penetrating peptide on the expression of inflammatory factor mRNA is compared with that of LPS stimulation group, n=3.

FIG. 8 is a photograph showing TAT, TAT-HuR-HNS-3 and olaparib are added to the cell culture medium 0.5 h in advance. The expression of CXCL2 mRNA is detected by Realtime PCR after TNF-α has stimulated RAW264.7 cells for 1 hour. The working concentration of cell penetrating peptide is 25 μM. **P<0.001, the effect of cell penetrating peptide on the expression of inflammatory factor mRNA is compared with that of LPS stimulation group, n=3.

FIG. 9 is a photograph showing TAT, TAT-HuR-HNS-3 and olaparib are added to the cell culture medium 0.5 h in advance. The expression of IL-1β mRNA is detected by Realtime PCR after TNF-α has stimulated RAW264.7 cells for 1 hour. The working concentration of cell penetrating peptide is 25 μM. **P<0.001, the effect of cell penetrating peptide on the expression of inflammatory factor mRNA is compared with that of LPS stimulation group, n=3.

FIG. 10 is a photograph showing TAT, TAT-HuR-HNS-3 and olaparib are added to the cell culture medium 0.5 h in advance. The expression of TNF-α mRNA is detected by Realtime PCR after TNF-α has stimulated RAW264.7 cells for 1 hour. The working concentration of cell penetrating peptide is 25 μM. **P<0.001, the effect of cell penetrating peptide on the expression of inflammatory factor mRNA is compared with that of LPS stimulation group, n=3.

FIG. 11 is a photograph showing TAT, TAT-HuR-HNS-3 and olaparib are added to the cell culture medium 0.5 h in advance. The expression of CXCL2 mRNA is detected by Realtime PCR after LPS has stimulated RAW264.7 cells for 1 hour. The working concentration of cell penetrating peptide is 25 μM. **P<0.01, the effect of cell penetrating peptide on the expression of inflammatory factor mRNA is compared with that of LPS stimulation group, n=3.

FIG. 12 is a photograph showing TAT, TAT-HuR-HNS-3 and olaparib are added to the cell culture medium 0.5 h in advance. The expression of IL-1β mRNA is detected by Realtime PCR after LPS has stimulated RAW264.7 cells for 1 hour. The working concentration of cell penetrating peptide is 25 μM. **P<0.01, the effect of cell penetrating peptide on the expression of inflammatory factor mRNA is compared with that of LPS stimulation group, n=3.

FIG. 13 is a photograph showing TAT, TAT-HuR-HNS-3 and olaparib are added to the cell culture medium 0.5 h in advance. The expression of TNF-α mRNA is detected by Realtime PCR after LPS has stimulated RAW264.7 cells for 1 hour. The working concentration of cell penetrating peptide is 25 μM. **P<0.01, the effect of cell penetrating peptide on the expression of inflammatory factor mRNA is compared with that of LPS stimulation group, n=3.

FIG. 14 is a photograph showing the inhibition of the short peptide TAT-HuR-HNS-3 on the accumulation of neutrophils in the lungs of mice. The mice of Saline group are administered normal saline by intranasal administration after anesthesia. The mice of LPS group are administered LPS by intranasal administration after anesthesia. The mice of LPS+HuR-HNS-3 group are administered TAT-HuR-HNS-3 short peptide by intranasal administration after anesthesia and administered LPS 1 h after administrating TAT-HuR-HNS-3 short peptide, and the mice of all group are sacrificed to death at 16 h after intranasal administration. The alveolars lavage fluid of each group is extracted, smeared and stained with Wright-Giemsa. The alveolar lavage fluid of mice after LPS stimulation contains a large number of neutrophils. Pretreatment of the mice with cell penetrating peptide TAT-HuR-HNS-3 may significantly reduce the number of neutrophils.

FIG. 15 is a photograph showing the inhibition of the short peptide TAT-HuR-HNS-3 on the inhibition of the expression of inflammatory factors in the lungs of mice. The mice of Saline group are administered normal saline by intranasal administration after anesthesia. The mice of LPS group are administered LPS by intranasal administration after anesthesia. The mice of LPS+HuR-HNS-3 group are administered TAT-HuR-HNS-3 short peptide by intranasal administration after anesthesia and administered LPS 1 h after administrating TAT-HuR-HNS-3 short peptide, and the mice of all group are sacrificed to death at 1 h after intranasal administration. The total RNA of lung tissue is extracted and reversely transcribed, then the inflammatory factor mRNA is detected after reverse transcription, the result of real time PCR shows that compared with LPS group, the mRNAs of CCL2, CXCL2, IL-1β and TNF-α in the LPS+HuR-HNS-3 group are significantly decreased. ***P<0.001. The effect of cell penetrating peptide on the expression of inflammatory factor mRNA is compared with that of LPS stimulation group, n=3.

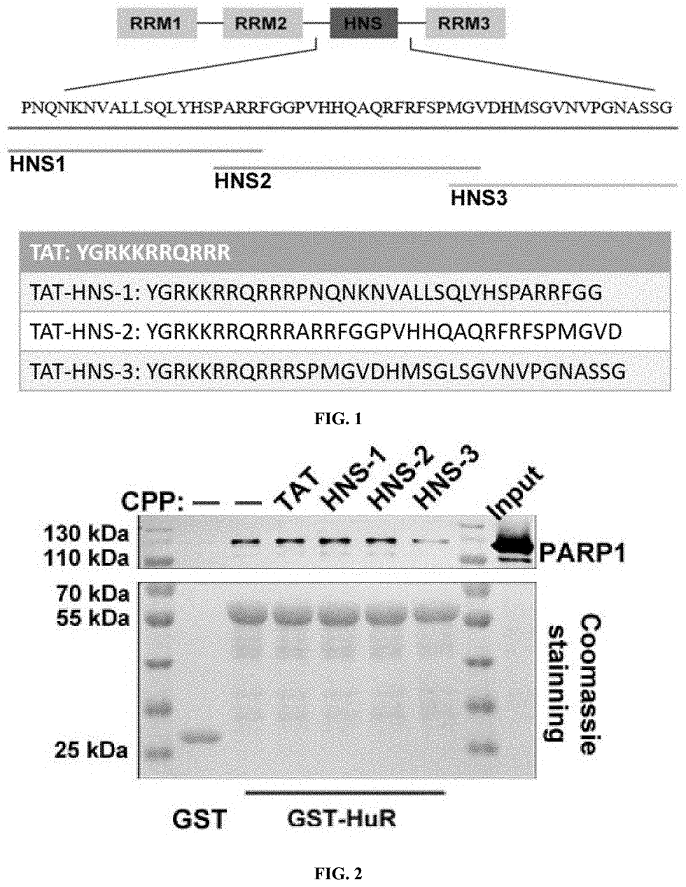

### DETAILED DESCRIPTION OF THE EMBODIMENTS

The present disclosure will be further described below in combination with the examples.

### Example 1 Materials

1. Cell Strain

Human renal epithelial cells HEK293, mouse macrophage cells RAW264.7, mouse alveolar epithelial cells MLE12 from the laboratory cell bank which was purchased from ATCC.

2. Reagents, Antibodies and Enzymes

| Drug Name | Item No. | Purchase company |
|---|---|---|
| TNF-α | 300-01A | PeproTech |
| LPS | L2630 | Sigma |
| Olaparib | AZD2281 | SELLECT |
| Anti-His antibody | HT501 | TransGen Biotech |
| Anti-GFP antibody | HT801 | TransGen Biotech |
| Anti-HuR antibody | 3A2, sc-5261 | Santa Cruz Biotechnology |
| Anti-PARP1 antibody | B-10, sc-74470 | Santa Cruz Biotechnology |

3. Medium and Primary Reagent

A. DMEM cell culture medium (2 L): Commercial DMEM medium, 7.4 g NaHCO₃.

PBS(1 L) containing 8 g NaCl, 0.2 g KCl, 3.576 g Na₂HPO₄.12H₂O, 0.24 g KH₂PO₄ was used after packaging and sterilization.

B. Whole cell lysate (protosalt): 150 mM NaCl, 20 mM Tris (pH7.5), 1 mM EDTA, 1 mM EGTA, 1% NP-40, 2.5 mM sodium pyrophosphate, 1 mM β-glycerophosphate, 1 mM Na₃VO₄, 5 μg/mL aprotinin/leupeptin

C. Western Blot Related Reagents

a. 10× running buffer (1 L): 30 g Tris, 144 g glycine, 10 g SDS;

1× running buffer (500 mL): 50 mL 10× running buffer, 450 mL double distilled water.

b. 10× transfer buffer (1 L): 30.3 g Tris, 144 g glycine;

1× transfer buffer (1 L): 100 mL 10× transfer buffer, 900 mL double distilled water.

c. 10×TBS (1 L): 24.2 g Tris, 80 g NaCl;

1×TBST (1 L): 100 mL 10×TBS, 900 mL double distilled water, 1 mL Tween, 700 μL concentrated hydrochloric acid.

d. Stripping Buffer (100 mL): 6.88 mL β-mercaptoethanol, 20 mL 10% SDS, 12.5 mL 0.5 M Tris-HCl (PH6.8);

e. Confining liquid (5% non-fat milk): 1 g skimmed milk powder, 20 mL TB ST.

f. 2× Loading Buffer (100 mL): 20 mL 0.5 M Tris-HCl (PH6.8), 20 mL glycerol, 40 mL 10% SDS, 0.1 g 0.1% bromophenol blue, 10 mL β-mercaptoethanol.

g. Coomassie brilliant blue staining solution (2 L): 1 g Coomassie brilliant blue R250, 900 mL methanol, 900 mL double distilled water, 200 mL glacial acetic acid.

h. Protein destaining solution (500 mL): 50 mL methanol, 50 mL glacial acetic acid, 400 ml double distilled water.

i. Preparation method of protein adhesives:

Separation Gel

| Gel | Double | Acrylamide | PH 8.8 | 10% | Ammonium | TEMED |
|---|---|---|---|---|---|---|
| concentration | distilled water (mL) | (mL) | Tris-HCl (mL) | SDS (μL) | persulfate (μL) | (μL) |
| 9% | 2.2 | 1.5 | 1.25 | 50 | 25 | 5 |
| 10% | 2.05 | 1.65 | 1.25 | 50 | 25 | 5 |
| 11% | 1.85 | 1.85 | 1.25 | 50 | 25 | 5 |

Spacer Gel

| | Double | Acrylamide | PH 6.8 | 10% | Ammonium | TEMED |
|---|---|---|---|---|---|---|
| | distilled water (mL) | (mL) | Tris-HCl (mL) | SDS (μL) | persulfate (μL) | (μL) |
| Plate 1 | 1.425 | 0.425 | 0.625 | 25 | 12.5 | 2.5 |
| Plate 2 | 2.85 | 0.85 | 1.25 | 50 | 25 | 5 |

4. Induction and Purification of Protein and GST-Pull Down Related Reagents:

A. Solution for replacement: 50 mM Tris-HCl (pH>8.0), 100 mM KCl

B. Eluent solution: 1 mL solution for replacement+40 mM reduced glutathione (add PMSF when used)

C. Bacteria lysate:

| | 25 mL | 100 mL |
|---|---|---|
| Hepes (μL) | 500 | 20000 |
| NaCl (μL) | 960 | 2769 |
| 50% glycerol (mL) | 5 | 20 |
| EDTA (μL) | 100 | 400 |
| Leu + Apr (μL) | 10 | 40 |
| PMSF(μL) | 25 | 100 |
| H₂O (mL) | 18.42 | 73.68 |

D. TEN100: 20 mM Tris(pH7.5), 0.1 mM EDTA, 100 mM NaCl.

E. NETN: 20 mM Tris(pH7.5), 0.1 mM EDTA, 100 mM NaCl, 0.5% NP-40.

5. Conventional PCR

β-actin primer sequence (Mouse origin):
Forward primer (F) (SEQ ID NO. 5):
5′-AACAGTCCGCCTAGAAGCAC-3′,
Reverse primer (R) (SEQ ID NO. 6):
5′-CGATGACATCCGTAAAGACC-3′;
CXCL-2 primer sequence:
Forward primer (F) (SEQ ID NO. 7):
5′-TCAATGCCTGAAGACCC-3′,
Reverse primer (R) (SEQ ID NO. 8):
5′-TGGTTCTTCCGTTGAGG-3′;
IL-1β primer sequence:
Forward primer (F) (SEQ ID NO. 9):
5′-CAGGATGAGGACATGAGCAC-3′,
Reverse primer (R) (SEQ ID NO. 10):
5′-CTCTGCAGACTCAAACTCCAC-3′;
TNF-α primer sequence:
Forward primer (F) (SEQ ID NO. 11):
5′-TACTGAACTTCGGGGTGATTGGTCC-3′,
Reverse primer (R) (SEQ ID NO. 12):
5′-CAGCCTTGTCCCTTGAAGAGAACC-3′;
CCL2 primer sequence:
Forward primer (F) (SEQ ID NO. 13):
5′-AAGTTGACCCGTAAATCTGA-3′,
Reverse primer (R) (SEQ ID NO. 14):
5′-TGAAAGGGAATACCATAACA-3′;

Experiment Instrument

Dehydration shaker and horizontal shaker (Beijing Liuyi), electrophoresis apparatus and membrane transfer device (Bio-Rad), CO₂ cultivation box (Thermo Forma 3111), PCR instrument (Thermo Electron Corporation), confocal laser scanning microscope (Zeiss), sigma high speed low temperature centrifuge, ultrasonic cell disruptor (Ningbo Machinery Factory), Xingyu DAKW-D bain-marie, ND-1000 spectrometer, fluorescence quantitative PCR instrument, high speed refrigerated centrifuge (Beckman Coulter), ultra-clean bench (Air Tech, Suzhou Purification Instrument Factory), automatic chemiluminescence image analysis system, vortex genie (Vortex genie-2, Scientific industries), etc., are used.

Experimental Method is Shown in FIG. 2:

1. Induced Expression and Purification of GST-Tagged Protein

a. Bacterial activation: The night before, the strain with GST and GST-HuR was activated at a ratio of 1:100. 40 μL strain was added to 4 ml LB culture medium, and cultured overnight in incubator with shaking at 37° C.

b. Induced expression of protein: The bacteria solution cultured overnight was amplified according to the ratio of 1:8. 4 ml of bacteria solution was added into 28 ml LB medium and cultured in incubator at 37° C. for 1 h. 64 μL 0.5 M IPTG and 64 μL 20 mM ZnSO₄ were added as protein expression inducer. The bacteria solution was cultured in incubator at 37° C. for 3-4 h.

c. Equilibrium of beads: 200 μL reduced glutathione 4B beads were added in 800 μL PBS. Then PBS was centrifuged at 4° C., 500 G for 5 min, and the supernatant was discarded. Then 800 μL PBS was added to suspend the beads. The beads were equilibrated in the rotator at 4° C. for 1 h and centrifuged at 4° C., 500 G for 5 min. The supernatant was discarded.

d. Ultrasonic cracking bacteria: the bacteria was collected and centrifuged at 4° C., 4000 rpm for 10 min; the supernatant was discarded, the bacteria lysate was added (7.2 mL bacteria lysate was added in 32 mL bacteria solution), the bacteria was fully pipetted into a suspension. The bacteria was cracked by ultrasonic disruptor. The conditions of ultrasound were as follows:

| | | |
|---|---|---|
| Total time | 3 | min |
| Ultrasonic time | 9 | s |
| Intermittent time | 6 | s |
| Temperature protection | 15° | C. |
| Power | 15 | W |

The ultrasonic bacterial solution was translucent, which was divided and centrifuged at 4° C., 12000 rpm for 25 min. The supernatant was taken, and 10% NP40 was added. The final concentration of NP40 was 0.5%.

2. Purification of Protein

A. The equilibrated reduced glutathione 4B beads were incubated with the supernatant of the bacterial lysate on a rotator at 4° C. for 3 h or overnight. The centrifuge tube was sealed with parafilm to avoid leakage.

B. Wash of the beads: The beads were respectively washed twice with NETN and TEN100, in which 1 ml NETN/TEN100 was used for each time. The centrifugal conditions were: 4° C., 500 G, 5 min. In the meantime, the beads were transferred to a 1.5 EP tube. Then the beads were suspended with TEN100 and equilibrated in a rotator at 4° C. for 1 h. The beads were preserved with PBS after the supernatant was discarded.

3. GST Pull-Down Experiment

A. Extraction of whole cell lysate.

B. The GST, GST-HuR and the extracted cell lysate were incubated at 4° C. for 3 h or overnight. In this process, three short peptides, HuR-HNS-1, HuR-HNS-2 and HuR-HNS-3 were added separately.

C. TEN100 was used to wash 4 times to remove impurity protein. The supernatant was discarded after centrifugation. 2× loading buffer equal to the volume of the beads was added, and the extracted cell lysate was boiled for 5 minutes.

D. 10 μl of protein was taken and loaded, and the interaction of protein was detected by protein electrophoresis and Western blot.

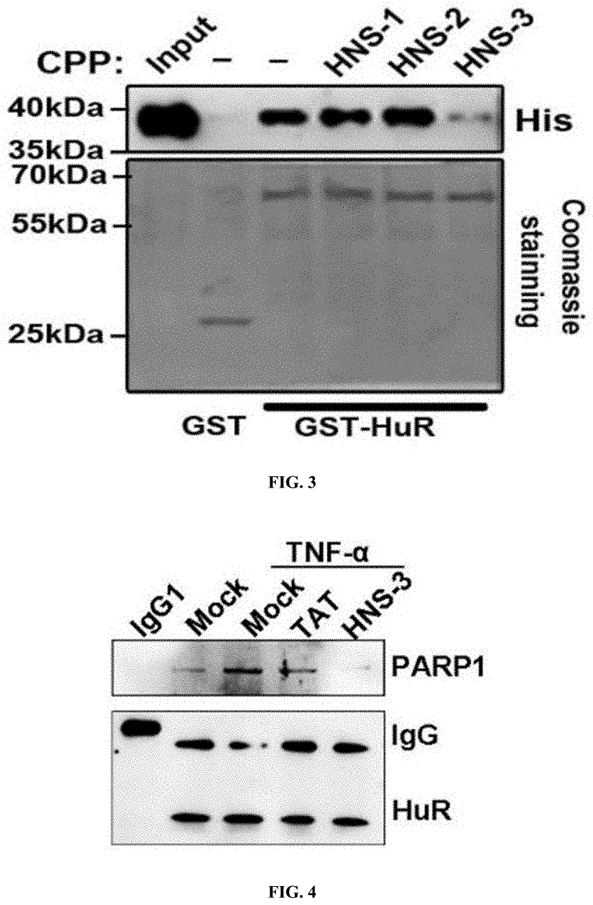

As shown in FIG. 3:

1. Induced Expression and Purification of GST/HIS-Tagged Protein

A. Bacterial activation: The night before, the strain with GST, GST-HuR and His-HuR was activated at a ratio of 1:100. 40 μL strain was added to 4 ml LB culture medium, and cultured overnight in incubator with shaking at 37° C.

B. Induced expression of protein: The bacteria solution cultured overnight was amplified according to the ratio of 1:8. 4 ml of bacteria solution was added into 28 ml LB medium and cultured in incubator at 37° C. for 1 h. 64 μL 0.5 M IPTG and 64 μL 20 mM ZnSO₄ were added as protein expression inducer. The bacteria solution was cultured in incubator at 37° C. for 3-4 h.

C. Equilibrium of beads: 200 μL reduced glutathione 4B beads were added in 800 μL PBS. Then PBS was centrifuged at 4° C., 500 G for 5 min. The supernatant was discarded. Then 800 μL PBS was added to suspend the beads. The beads were equilibrated in the rotator at 4° C. for 1 h and centrifuged at 4° C., 500 G for 5 min. The supernatant was discarded.

D. Ultrasonic cracking bacteria: the bacteria was collected and centrifuged at 4° C., 4000 rpm for 10 min; the supernatant was discarded, the bacteria lysate was added (7.2 mL bacteria lysate was added in 32 mL bacteria solution), the bacteria was fully pipetted into a suspension. The bacteria was cracked by ultrasonic disruptor. The conditions of ultrasound were as follows:

| | | |
|---|---|---|
| Total time | 3 | min |
| Ultrasonic time | 9 | s |
| Intermittent time | 6 | s |
| Temperature protection | 15° | C. |
| Power | 15 | W |

The ultrasonic bacterial solution was translucent, which was divided and centrifuged at 4° C., 12000 rpm for 25 min. The supernatant was taken, and 10% NP40 was added. The final concentration of NP40 was 0.5%.

2. Purification of GST and GST-HuR Protein

A. The equilibrated reduced glutathione 4B beads were incubated with the supernatant of the bacterial lysate on a rotator at 4° C. for 3 h or overnight. The centrifuge tube was sealed with parafilm to avoid leakage.

B. Wash of the beads: The beads were washed respectively twice with NETN and TEN100, in which 1 ml NETN/TEN100 was used for each time. The centrifugal conditions are: 4° C., 500 G, 5 min. In the meantime, the beads were transferred to a 1.5 EP tube. Then the beads were suspended with TEN100 and equilibrated in a rotator at 4° C. for 1 h. The beads were preserved with PBS or eluted continuously after the supernatant was discarded.

C. Elution of the protein: 1 mL of solution for replacement was added to suspend the beads. The beads equilibrated and washed twice with the solution for replacement. 150 μl of eluent was added after discarding the supernatant, and eluted in a shaker for 1 h at room temperature. During this period, the bottom of the tube was flicked several times to make it fully eluted. After centrifugation, the supernatant was the eluent and transferred to a new 1.5 ml EP tube. 4B beads with protein and protein eluate were conserved at −80° C. for a long-time reservation, repeated freezing and thawing is preferably avoided.

D. The protein content after purification was detected by Coomassie Brilliant Blue R250 staining

3. Pull-Down Experiment

A. The His-HuR, eluted GST and GST-HuR protein were incubated at 4° C. for 3 h or overnight. In this process, three short peptides, HuR-HNS-1, HuR-HNS-2 and HuR-HNS-3 were added separately.

B. TEN100 was used to wash 4 times to remove impurity protein. The supernatant was discarded by centrifugation. 2× loading buffer equal to the volume of the beads was added, and the extracted cell lysate was boiled in boiling water for 5 minutes.

C. 10 μL of protein was took and loaded, and the interaction of protein was detected by protein electrophoresis and Western blot.

As shown in FIG. 4:

1. TAT, TAT-HuR-HNS-3 were added to the cell culture medium 0.5 h in advance. HEK293 cells were stimulated with TNF-α for 1 h.

2. The whole cell lysates of different stimulation groups were extracted, and immunoprecipitation experiments were performed with HuR antibody and magnetic beads.

3. The whole cell lysate of low, medium and high salt was respectively used once to wash and remove the impurity protein. The supernatant was discarded after centrifugation, 2× loading buffer equal to the volume of the beads was added, and the sample was boiled in boiling water for 5 min.

4. 10 μL protein samples were taken and loaded, and the interaction of protein was detected by protein electrophoresis and Western blot.

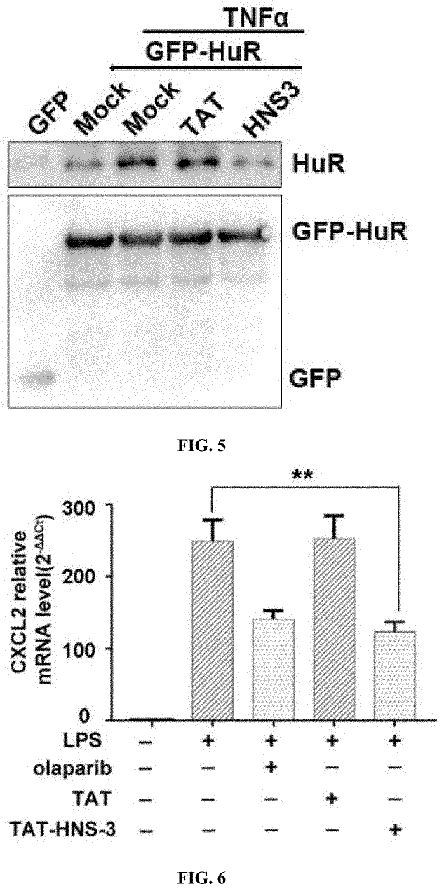

As shown in FIG. 5:

1. GFP and GFP-HuR were transfected into HEK293 cells, At 6 h post-transfection, cells were replaced with complete medium to promote recovery.

2. After 24 hours of transfection, TAT and TAT-HuR-HNS-3 were added to the cell culture medium 0.5 h in advance, and HEK293 cells were stimulated with TNF-α for 1 h.

3. The whole cell lysates of different stimulation groups were extracted, and immunoprecipitation experiments were performed with HuR antibody and magnetic beads.

4. The whole cell lysate was washed with low, medium and high salt respectively once to remove the impurity protein. The supernatant was discarded after centrifugation, 2× loading buffer equal to the volume of the beads was added, and the sample was boiled in boiling water for 5 min.

5. 10 μL protein samples were taken and loaded, and the interaction of protein was detected by protein electrophoresis and Western blot.

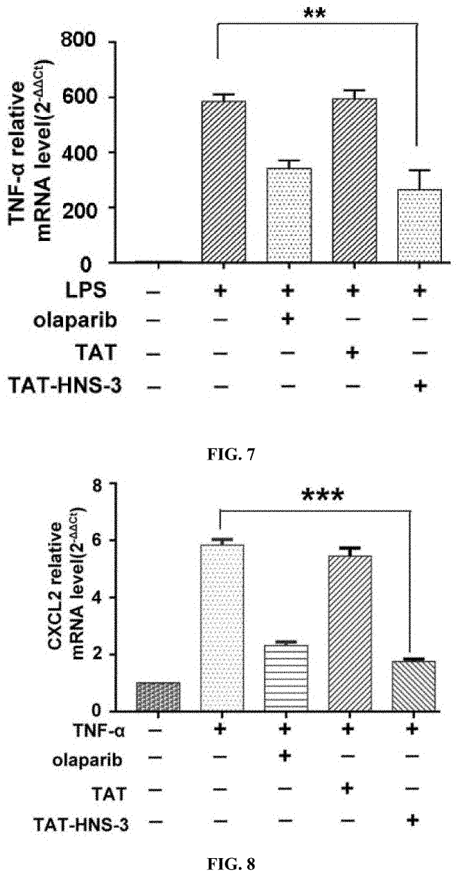

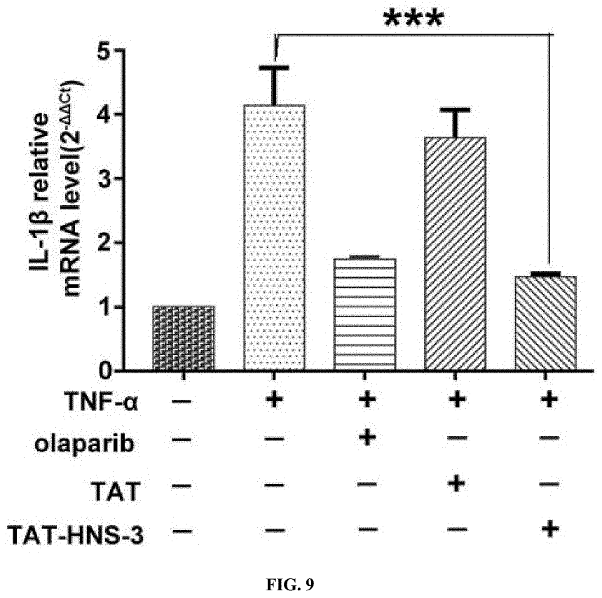

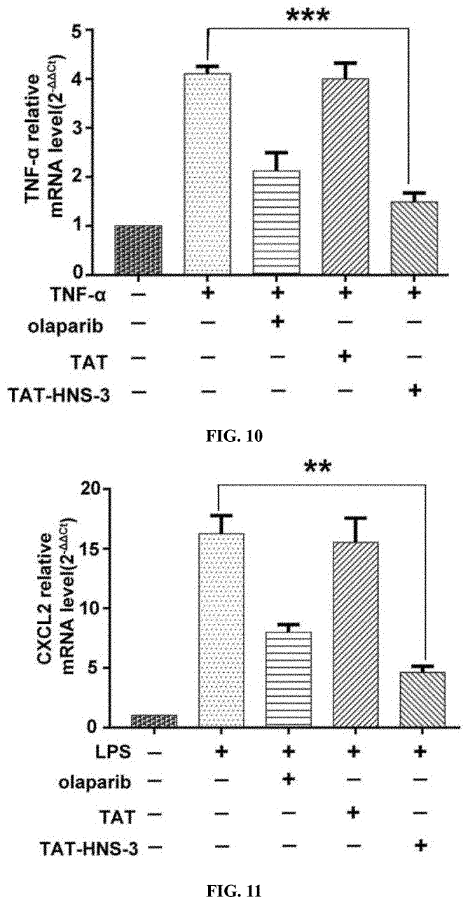

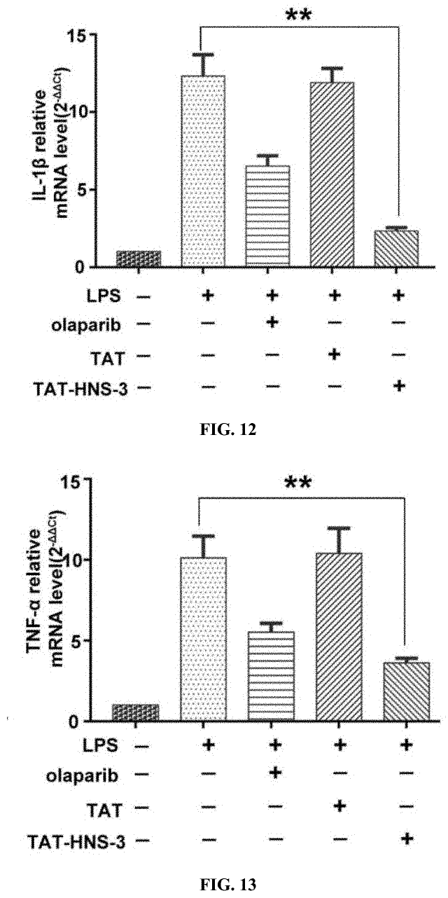

As shown in FIGS. 6-13, the expression of inflammatory factors mRNA was detected

1. Treatment of Cells

TAT and TAT-HuR-HNS-3 were added to the cell culture medium 0.5 h in advance. MLE12 or RAW264.7 cells were stimulated respectively with TNF-α and LPS for 1 h.

2. The Extraction of Cell RNA

RNA-free tip, 1.5 ml EP centrifuge tube and reagent were selected to extract RNA. 6-well plate was taken as an example, the specific process was as follows:

A. Collecting cells: The treated cells were taken and the medium was discarded, then the cells were washed with precooled PBS, and the PBS was discarded. 1 mL of Trizol was added, the cells was fully pipetted until clear, and transferred to the corresponding 1.5 mL EP tube.

B. Separation of RNA: the nucleic acid and protein were separated after 10 min at room temperature. 200 μL of trichloromethane was added. The mixture was shaken violently for 1 min, and placed still at room temperature for 3 min, and centrifuged at 12000 rpm for 5 min. RNA was in the upper phase.

C. Precipitation of RNA: 400 μL of upper liquid was transferred into a new 1.5 mL EP tube, 400 μL isopropanol was added. The mixture was shaken violently for 1 min, and placed at room temperature for 1 min or −20° C. overnight. RNA precipitation was observed after centrifugation at 12000 rpm for 5 min.

D. Washing of RNA: The supernatant was discarded, 1 mL of 70% ethanol was added. The mixture was shaken, mixed and centrifugated at 12000 rpm for 5 min. The supernatant was discarded and only RNA precipitation was left. RNA precipitation was dried at room temperature for a few minutes until there was no residual ethanol. Note: RNA could not be dried to transparent color.

E. Dissolution of RNA: the dried RNA precipitation was added to corresponding volume of DEPC water (20-30 μL) and dissolved for about 10 min. The concentration of RNA was detected with ND-1000. OD260/OD280>1.9 indicated that RNA purity was high and impurities were less. Because of the instability of RNA, it was put on ice in time or conserved in refrigerator at −80° C. for a long-time reservation.

3. Reverse Transcription-Polymerase Chain Reaction (RT-PCR)

The total RNA extracted from the cells was reverse transcribed into cDNA. The specific steps were as follows:

A. The total RNA was placed in water bath at 42° C. for 5 min;

| | |
|---|---|
| Total RNA | 1 μL |
| RNAase free H₂O | 7 μL with RNA volume |
| gDNA eraser | 1 μL |
| 5 × DNA eraser buffer | 2 μL |

B. The total RNA was placed in water bath at 42° C. for 15 min; then placed in water bath at 85° C. for 5 s.

| | |
|---|---|
| Primer sc RT en mx | 1 μL |
| RT primer mix | 1 μL |
| 5 × primer buffer | 4 μL |
| RNase free H₂O | 4 μL |

4. Real Time PCR (Quantitative Real-Time PCR)

PCR products were quantitative analyzed, PCR products were detected by using fluorescence energy transfer technology, with adding off fluorescent labeled probes and using of fluorescent signals. The cDNA was diluted proportionally in advance. After sample loading, fluorescence quantitative PCR was used to detect the sample and the data is analyzed afterwards. The PCR system (25 μL) was as follows:

| | | |
|---|---|---|
| cDNA | 2 | μL |
| SYBR | 12.5 | μL |
| Rox | 0.5 | μL |
| Sterile water | 8 | μL |
| Primer | 2 | μL |

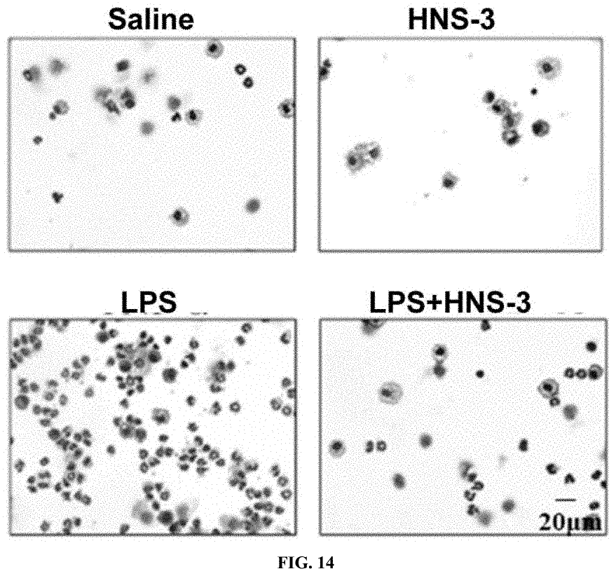

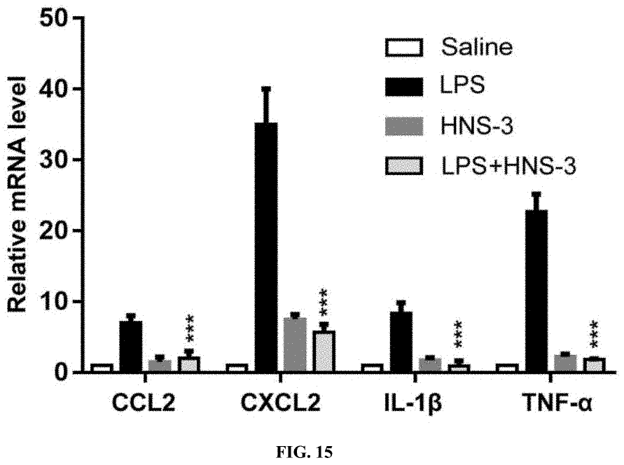

As shown in FIGS. 14-15:

1. Anesthesia of Mice

Pentobarbital sodium was injected intraperitoneally into the mice after grasping according to the standard posture (the mice entered coma state after 3 minutes, and the coma lasted for about 40 minutes).

2. Intranasal Administration in Mice

The anaesthetized mice were grasped for intraperitoneal injection, and the head was adjusted in the upward state. TAT-HuR-HNS-3, LPS or Saline was gently dropped (about 15 uL per drop) into the nostrils of mice with a pipette, and then TAT-HuR-HNS-3, LPS and Saline were added slowly according to the left and right nostrils in turn (the liquid quickly inhaled into the nasal cavity with the mice breathing) and the drops were kept for about one minute.

C. Extraction of Bronchoalveolar Lavage Fluid (BALF) from Mice

The mice were put to death by injecting EDTA into tail vein. The mice were fixed on the anatomical table in supine position. The skin of neck and chest was cut off with scissors, and the muscle tissue around the organs was stripped to expose the trachea (pay attention to avoid cutting the blood vessels in the neck). A needle was inserted from the mouth, the needle was gently fed down the trachea until the needle reached the end of the trachea. The trachea with the needle inserted was tied tightly with surgical thread to prevent the needle from sliding in the trachea during the subsequent operation. The syringe with a pre cooled PBS was connected to the needle. the PBS was slowly and completely injected, and then slowly sucked (suction is more difficult than injection, the bulge and contractic of pleural were observed in the whole process), and there was a large amount of white foamy substance in the liquid sucked. The liquid sucked was transferred to a 1.5 mL EP tube and conserved on ice for use.

D. Observation of Staining in Alveolar Extract Cells

Bronchoalveolar lavage fluid (BALF) included cell secretions such as a variety of cells and soluble proteins. The cells were separated from other small molecules by centrifugation at 2 000 rpm/min at 4° C. for 5 min. Cells were located in the precipitation, and soluble proteins and other molecules were in the supernatant. There may be red blood cells in bronchoalveolar lavage fluid due to lung injury caused by stimulation or injury during operation. Red blood cells were cracked for the purpose of follow-up experiments. The supernatant of bronchoalveolar lavage fluid was discarded after centrifugation. The red blood cell lysate was added to suspend the cells. The cells were repeatedly pipetted until uniformity, placed at room temperature for 2 min, and centrifuged at 300 g/min for 10 min (it may be repeated several times according to the specific cracking conditions).

E. Cell Smear, Staining

a. The cells of the bronchoalveolar lavage fluid with red blood cells removed were resuspended with PBS, mixed, and 2-fold diluted.

b. Appropriate amount of diluted cell suspension was taken and dropped on the clean slide, and spread gently with the cover glass.

c. Pure methanol was dripped to fix for at least 30 s after the water on the slide evaporated.

d. The slide was tilted, the methanol was removed, the slide was placed flat, appropriate amount of staining solution was added, the cells were stained for 2 min.

e. The equal amount of Sorensen's buffer was dropped and mixed with the staining solution gently. The slide was not touched (the surface of reaction liquid showed metallic luster). The function of the staining solution was lasted for 3 min.

f. The slides were rinsed gently with ultrapure water for 30 s. The slides were tilted and dried (the slides were not dried with filter paper).

g. Observation (cell classification, counting).

F. Extraction of Mice Lung Tissue

After the mice were sacrificed by injection EDTA into tail vein, the mice were dissected. The lung was removed, and washed twice in cooled PBS. The water on the surface was sucked dry by filter paper, and the lung was placed on ice for use.

G. Extraction of RNA from Mice Lung

a. Homogenation of tissue: 100 mg lung tissue was weighed, put into culture dish and cut on ice. The lung tissue was suspended with 3 mL cooled PBS and transferred to a grinder. The lung tissue was transferred to a centrifuge tube after full grinding and centrifuged at 4° C., 1200 rpm/min for 5 min, and the supernatant was discarded. The precipitation was suspended by 1 mLTrizol. The cells were repeatedly pipetted until uniformity, transferred to 1.5 ml EP tube. The cells were repeatedly pipetted and transferred to 1.5 ml EP tube.

b. Separation of RNA: 1.5 ml EP tube was inverted and mixed uniformly, and placed at room temperature for 10 min to separate nucleoprotein and RNA; 200 μL chloroform (trichloromethane) was added, and gyre oscillation was performed for 90 s, and then the mixture was placed at room temperature for 3 min and centrifugated at 4° C., 12000 rpm/min for 5 min. The liquid in the EP tube was observed to be divided into two layers, and the upper layer was RNA.

The following steps were operated with RNase-free EP tube and tip

c. RNA precipitation: about 400 μL of the upper water phase with RNA was transferred into the marked 1.5 mL RNase-free EP tube (the liquid surface of the lower layer was not sucked to prevent protein contamination), the equal volume of isopropanol was added (about 400 μL), mixed uniformly (the upper water phase was shaken gently to see whether it was uniformly mixed according to refractivity), and the upper water phase was placed at room temperature for 10 min, and centrifugated at at 4° C., 12000 rpm/min for 10 min. The supernatant was pipetted (the precipitation was not discarded).

d. Washing of RNA: ethanol was added into EP tube of step c according to the proportion of adding at least 1 mL 70% ethanol (prepared by DEPC treated water) per mL of Trizol. The EP tube was gently oscillated to make RNA precipitation suspend without adhering to the wall; the mixture was centrifuged at 4° C., 12000 rpm/min for 10 min; then the supernatant was discarded.

e. The step d was repeated. Centrifugation was performed, and the residual liquid was oscillated with a pipette.

f. Dissolution of RNA: after washing, the RNA was dried for 5-10 min with the cap opened at room temperature (a prefer status is that the edge of the precipitate tended to be transparent. Note: the drying should be terminated before the precipitation was completely transparent.). 10-20 μL DEPC water was added to dissolve RNA according to the volume of precipitation. The precipitation was pipetted gently for 8-10 times, and placed at room temperature for 10-20 min to dissolve RNA completely, and conserved at −80° C. for use.

g. RNA concentration was measured by ND-1000 spectrometer.

H. Reverse Transcription-Polymerase Chain Reaction (RT-PCR)

The total RNA extracted from the cells was reverse transcribed into cDNA. The specific steps were as follows:

a. The total RNA was placed in water bath at 42° C. for 5 min;

| | |
|---|---|
| Total RNA | 1 μL |
| RNAase free H₂O | 7 μL with RNA volume |
| gDNA eraser | 1 μL |
| 5 × DNA eraser buffer | 2 μL |

b. The total RNA was placed in water bath at 42° C. for 15 min; then placed in water bath at 85° C. for 5 s.

| | |
|---|---|
| Primer sc RT en mx | 1 μL |
| RT primer mix | 1 μL |
| 5 × primer buffer | 4 μL |
| RNase free H₂O | 4 μL |

I. Real Time PCR (Quantitative Real-Time PCR)

PCR products were quantitative analyzed, PCR products were detected by using fluorescence energy transfer technology, with adding of fluorescent labeled probes and using of fluorescent signals. The cDNA was diluted proportionally in advance. After sample loading, fluorescence quantitative PCR was used to detect the sample and the data was analyzed afterwards. The PCR system (25 μL) was as follows:

| | | |
|---|---|---|
| cDNA | 2 | μL |
| SYBR | 12.5 | μL |
| Rox | 0.5 | μL |
| Sterile water | 8 | μL |
| Primer | 2 | μL |

### REFERENCE

[1] Brennan C M, Steitz J A. HuR and mRNA stability [J]. Cell Mol Life Sci, 2001, 58(2): 266-277.
[2] Anderson P. Post-transcriptional regulons coordinate the initiation and resolution of inflammation [J]. Nat Rev Immunol, 2010, 10(1): 24-35.
[3] Hao S, Baltimore D. The stability of mRNA influences the temporal order of the induction of genes encoding inflammatory molecules [J]. Nat Immunol, 2009, 10(3): 281-288.
[4] Khabar K S. The AU-rich transcriptome: more than interferons and cytokines, and its role in disease [J]. J Interferon Cytokine Res, 2005, 25(1): 1-10.
[5] Costantino C L, Witkiewicz A K, Kuwano Y, et al. The role of HuR in gemcitabine efficacy in pancreatic cancer: HuR Up-regulates the expression of the gemcitabine metabolizing enzyme deoxycytidine kinase [J]. Cancer Res, 2009, 69(11): 4567-4572.
[6] Wang J G, Collinge M, Ramgolam V, et al. LFA-1-dependent HuR nuclear export and cytokine mRNA stabilization in T cell activation [J]. J Immunol, 2006, 176(4): 2105-2113.
[7] Winzen R, Gowrishankar G, Bollig F, et al. Distinct domains of AU-rich elements exert different functions in mRNA destabilization and stabilization by p38 mitogen-activated protein kinase or HuR [J]. Mol Cell Biol, 2004, 24(11): 4835-4847.
[8] Sugihara M, Tsutsumi A, Suzuki E, et al. Effects of infliximab therapy on gene expression levels of tumor necrosis factor alpha, tristetraprolin, T cell intracellular antigen 1, and Hu antigen R in patients with rheumatoid arthritis [J]. Arthritis Rheum, 2007, 56(7): 2160-2169.
[9] Suzuki E, Tsutsumi A, Sugihara M, et al. Expression of TNF-alpha, tristetraprolin, T-cell intracellular antigen-1 and Hu antigen R genes in synovium of patients with rheumatoid arthritis [J]. Int J Mol Med, 2006, 18(2): 273-278.
[10] Nieminen R, Vuolteenaho K, Riutta A, et al. Aurothiomalate inhibits COX-2 expression in chondrocytes and in human cartilage possibly through its effects on COX-2 mRNA stability [J]. Eur J Pharmacol, 2008, 587(1-3): 309-316.
[11] Esnault S, Malter J S. Hyaluronic acid or TNF-alpha plus fibronectin triggers granulocyte macrophage-colony-stimulating factor mRNA stabilization in eosinophils yet engages differential intracellular pathways and mRNA binding proteins [J]. J Immunol, 2003, 171(12): 6780-6787.
[12] Srikantan S, Gorospe M. HuR function in disease [J]. Front Biosci (Landmark Ed), 2012, 17(189-205.
[13] Luo X, Kraus W L. On PAR with PARP: cellular stress signaling through poly(ADP-ribose) and PARP-1 [J]. Genes Dev, 2012, 26(5): 417-432.
[14] Ke Y, Han Y, Guo X, et al. Erratum: PARP1 promotes gene expression at the post-transcriptional level by modulating the RNA-binding protein HuR [J]. Nat Commun, 2017, 8(15191.
[15] Filippova N, Yang X, Ananthan S, et al. Hu antigen R (HuR) multimerization contributes to glioma disease progression [J]. J Biol Chem, 2017, 292(41): 16999-17010.
[16] Scheiba R M, De Opakua A I, Diaz-Quintana A, et al. The C-terminal RNA binding motif of HuR is a multi-functional domain leading to HuR oligomerization and binding to U-rich RNA targets [J]. RNA Biol, 2014, 11(10): 1250-1261.
[17] Ke Y, Lv X, Fu X, et al. Poly(ADP-ribosyl)ation enhances HuR oligomerization and contributes to pro-inflammatory gene mRNA stabilization [J]. Cellular and Molecular Life Sciences, 2020.
[18] Schwarze S R, Hruska K A, Dowdy S F. Protein transduction: unrestricted delivery into all cells? [J]. Trends Cell Biol, 2000, 10(7): 290-295.
[19] Ramsey J D, Flynn N H. Cell-penetrating peptides transport therapeutics into cells [J]. Pharmacol Ther, 2015, 154(78-86).
[20] Wadia J S, Stan R V, Dowdy S F. Transducible TAT-HA fusogenic peptide enhances escape of TAT-fusion proteins after lipid raft macropinocytosis [J]. Nat Med, 2004, 10(3): 310-315.

1. A cell penetrating short peptide TAT-HuR-HNS-3, wherein the amino acid sequence of the cell penetrating short peptide TAT-HuR-HNS-3 for inflammatory diseases caused by the increase of inflammatory factors is as follows: YGRKKRRQRRR-SPMGVDHMSGLSGVNVPGNASSG.

2. The cell penetrating short peptide TAT-HuR-HNS-3 according to claim 1, wherein the amino acid sequence containing HNS-3 is used in the drug treating inflammatory diseases caused by the increase of inflammatory factors, wherein the inflammatory diseases comprising lung inflammation, asthma, pollen allergy, and nephritis.

3. The cell penetrating short peptide TAT-HuR-HNS-3 according to claim 1, wherein a delivery carrier connected to the amino acid sequence containing HNS-3 includes Pep-1, pVEC, MAP, pAntp, Transportan, Polyarginines.

4. The cell penetrating short peptide TAT-HuR-HNS-3 according to claim 1, wherein nanometer or small molecule materials are selected as a delivery carrier of the amino acid sequence containing HNS-3.

5. (canceled)

6. A method for treating inflammatory diseases of the lungs, wherein comprising administrating short peptide TAT-HuR-HNS-3 according to claim 1.

7. The method for treating inflammatory diseases of the lungs according to claim 6, wherein a delivery carrier connected to the amino acid sequence containing HuR-HNS-3 includes Pep-1, pVEC, MAP, pAntp, Transportan, Polyarginines.

8. The method for treating inflammatory diseases of the lungs according to claim 6, wherein nanometer or small molecule materials are selected as a delivery carrier of the amino acid sequence containing HuR-HNS-3.
